(19) United States
(12) Patent Application Publication
(10) Pub. No.: US 2008/0194642 A1
(43) Pub. Date: Aug. 14, 2008

(54) HIGH DOSE, LONG-ACTING ECTOPARASITICIDE FOR EXTENDED CONTROL

(75) Inventors: Robert Bruce Albright, Chalfont, PA (US); Shobhan Shashikant Sabnis, Pennington, NJ (US); Larry A. Kraft, Hightstown, NJ (US); Susan Joan Holzmer, Ewing, NJ (US); Robert Alan Pollet, Yardley, PA (US); Ramune Marija Cobb, Newton, PA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/028,219

(22) Filed: Feb. 8, 2008

Related U.S. Application Data
(60) Provisional application No. 60/889,261, filed on Feb. 9, 2007.
(60) Provisional application No. 60/966,832, filed on Aug. 30, 2007.

(51) Int. Cl. A61K 31/4439; A61K 31/175; A61P 33/14; A61K 31/415

(52) U.S. Cl. 514/341; 514/615; 514/407

(57) ABSTRACT

The present invention provides a topical, high-dose, long-acting ectoparasiticide composition, kit and method for protecting against ectoparasite infestations in a warm-blooded animal for a period of greater than about 6 weeks.

### CROSS-REFERENCE TO RELATED APPLICATIONS

This application claims the benefit under 35 U.S.C. § 119(e) to U.S. Provisional Patent Application No. 60/889,261, filed Feb. 9, 2007 and U.S. Provisional Patent Application No. 60/966,832, filed Aug. 30, 2007, the contents of which are hereby incorporated by reference in their entirety.

### BACKGROUND OF THE INVENTION

Arthropod ectoparasites commonly infecting warm-blooded animals include ticks, mites, lice, fleas, blowfly, the ectoparasite Lucilia sp. of sheep, biting insects including keds (Melophagus ovinus) and migrating dipterous larvae such as Hypoderma sp. and Dermatobia in cattle, Gastrophilus in horses and Cuterebra sp. in rodents, dogs and cats.

A number of treatments including metaflumizone, fipronil, pryiprole and imidacloprid are useful for the prevention and control of infestation by ectoparasites in warm-blooded animals. Topical administration (spot-on) is a preferred method for administering these compounds, which are limited in their duration of protection to approximately 4-6 weeks.

The limitations in duration of efficacy of these compounds is usually caused by loss of the active ingredient due to environmental or biological effects, including ruboff, degradation, and animal metabolism. Since the flea and tick seasons persist significantly longer than 4-6 weeks in many areas, multiple doses are required to achieve substantial protection from fleas and ticks for an entire season. Additionally, since ectoparasites can persist indoors during any climate, year-round protection is often preferred. It would therefore be desirable to formulate compositions that offer substantial protection from fleas and ticks, as well as other arthropods, for extended durations in a single application. Such applications would offer convenience, efficiency, and eliminate the risk of a gap in protection stemming from poorly-timed administration of additional doses.

Previously, high dose ectoparasiticide formulations have been avoided due to difficulties applying the material and instability of the active ingredient. Particularly, high volumes of conventional formulations resulted in product run-off and sodden appearances. Alternatively, high concentration formulations resulted in insolubility and degradation of the active ingredient, skin irritation as well as undesirable characteristics, such as poor viscosity, insufficient spreading, poor evaporation and inadequate permeation.

Furthermore, one cannot assume linearity in topical efficacy with changes in dose administered. In other words, an increase in dose does not necessarily imply a proportional increase in drug concentrations or in the degree of efficacy or in duration of activity. This is illustrated by drug pharmacokinetics. For example, saturation of pathways can limit the rate of absorption, metabolism or elimination of drugs and cause unexpected increases in concentration of drug in blood. In these cases, drug follows ‘zero-order kinetics, in what is known as Michaelis-Menten kinetics. Some well documented examples include ascorbic acid and naproxen. Differences in formulation, in route of administration, and physiological status can also impact the rate and extent of efficacy of drugs. Gibaldi & Perrier. Pharmacokinetics, Second Edition (1982), Marcel Deckker, Inc; M. Mayersohn (1972). Eur. J. Pharmac. 19:140; R. Runkel at al (1974) Clin Pharm Therap. 15:261. Particularly, the distribution, absorption, into the skin, subsequent resecretion into the hair, degradation and loss of topically applied insecticides can affect the duration of efficacy.

Thus, what is needed in the art are ectoparasiticide formulations and dosage regimens, which provide extended efficacy against parasitic infestations. Additionally, a need exists for topical ectoparasiticide formulations having a relatively high-dose or concentration.

Therefore it is an object of this invention to provide a high-dose, long-acting topical ectoparasiticide composition which is effective for protecting against ectoparasite infestation in a warm-blooded animal for a period of greater than about 6 weeks. It is another object of this invention to provide a method for the extension of the duration of activity of an ectoparasiticide in a warm-blooded animal. Further objects and features of the invention will become apparent from the description set forth hereinbelow.

### SUMMARY OF THE INVENTION

The present invention provides a high-dose, long-acting ectoparasiticide composition. Also provided is a method for extending the period of efficacy of an ectoparasiticide which comprises administering a high dose of said ectoparasiticide.

An additional aspect of the invention provides a method for preventing or treating an ectoparasite infestation in a warm-blooded animal for a period greater than 6 weeks, comprising:
topically administering to the warm-blooded animal a composition comprising an ectoparasiticide in a dose that is about 1.5 to 6 times the conventional dose for said ectoparasiticide.

Another aspect of the invention provides a composition which comprises on a weight to volume basis:
(a) about 5% to about 40% of ectoparasiticide;
(b) about 5% to about 25% of a bridging agent;
(c) about 0% to about 15% of a surfactant; and
(d) about 5% to about 80% of a carrier solvent or a mixture of solvents.

More particularly, the ectoparasiticide is a semicarbazone e.g. metaflumizone; a pyrazole e.g. fipronil, pyriprole; a neonicotinoid e.g. imidacloprid, dinotefuran; a pyrethroid e.g. permethrin, pyrethrins; an insect growth regulator e.g. pyriproxyfen, S-methoprene or a mixture thereof. More particular still, the composition comprises 26%-40% metaflumizone.

Another aspect of the invention provides a kit for preventing or treating an ectoparasite infestation in a warm blooded animal comprising a topical unit dose formulation of an ectoparasiticide comprising on a weight by volume basis:
(a) about 5% to about 40% of an ectoparasiticide;
(b) about 5% to about 25% of a bridging agent;
(c) about 0% to about 15% of a surfactant; and
(d) about 5% to about 80% of a carrier solvent or a mixture of solvents;
   wherein the unit dose comprises about 1.5 to 6 times the amount of the ectoparasiticide as compared to a conventional dose of said ectoparasiticide.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DETAILED DESCRIPTION OF THE INVENTION

Existing “spot-on” applications of topical veterinary ectoparasiticidal compositions applied to the base of the neck of the animal aid in making the applied composition difficult for the animal to remove, but require that a relatively small volume be applied. However, the solubility of ectoparasiticide formulations, particularly those containing metaflumizone, limits the ability to obtain high concentrations of ectoparasiticide in such applications.

Topical veterinary compositions containing metaflumizone as one of the active ingredients are highly desirable due to the effective and persistent activity of metaflumizone against a variety of ectoparasites, including, but not limited to, the cat flea, Ctenocephalides felis, and the dog flea, Ctenocephalides canis, in dogs or cats.

Surprisingly, it has now been found that ectoparasiticides, such as metaflumizone, may be formulated in a high-dose, long-acting, topical non-irritating composition comprising ectoparasiticide; a bridging agent or penetration enhancer, an optional surfactant, an optional polymeric agent and a carrier solvent or mixture of solvents. Accordingly, the present invention provides a high-dose, long-acting ectoparasiticide composition which comprises on a weight to volume basis:
(a) about 5% to about 40% of ectoparasiticide;
(b) about 5% to about 25% of a bridging agent;
(c) about 0% to about 15% of a surfactant; and
(d) about 5% to about 80% of a carrier solvent or a mixture of solvents.

In a particular embodiment, the ectoparasiticide is metaflumizone.

Advantageously, the composition of the invention retains the desired physical characteristics over time, without loss of potency of the active. Further, the composition of the invention exhibits sufficient viscosity, which allows for the retention of said composition when administered topically to an animal's skin or hair, and which facilitates the release of the ectoparasiticide, such as metaflumizone, over the desired extended period of time.

One aspect of the invention provides a method for preventing or treating an ectoparasite infestation in a warm-blooded animal for a period greater than 6 weeks, comprising:
topically administering to the warm-blooded animal a composition comprising an ectoparasiticide in a dose that is about 1.1 to 10 times the conventional dose for said ectoparasiticide.

In a more particular embodiment, the dose is 1.5 to 5 times the conventional dose for said ectoparasiticide. Alternatively, the dose is 1.2 to 5 times, 1.3 to 5 times or 1.4 to five times the conventional dose. More particularly, the dose is 2 to 5 times the conventional dose for said ectoparasiticide. More particular still, the dose is 2.5 to 4 times the conventional dose for said ectoparasiticide. More particular still, the dose is about three times the conventional dose for said ectoparasiticide. Alternatively, the dose is greater than 1.1, 1.2. 1.3, 1.4, 1.5, 1.6, 1.7, 1.8, 1.9, 2, 2.5 or 3 times the conventional dose.

In another embodiment, said period is from about 6 to about 30 weeks. More particularly, said period is from about 7 to about 20 weeks. More particular still, said period is from about 8 to about 20 weeks. More particularly, said period is from about 10 to about 20 weeks. More particularly, said period is from 12 to about 20 weeks. More particularly, said period is from about 14 to about 24 weeks. In another embodiment, said period is greater than about 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks or 20 weeks.

In another embodiment said ectoparasiticide is metaflumizone, fipronil, imidacloprid, pyriprole, dinotefuran or a mixture thereof. More particularly, said ectoparasiticide is metaflumizone. Alternatively, said ectoparasiticide is fipronil. Alternatively, said ectoparasiticide is imidacloprid. Alternatively, said ectoparasiticide is pyriprole and the composition further comprises butylhydroxytoluene. In another embodiment, the composition further comprises amitraz. More particularly, said animal is a dog, said ectoparasiticide is metaflumizone and the composition further comprises amitraz. In another embodiment, the composition further comprises methoprene. More particularly, said animal is a dog, said ectoparasiticide is fipronil and the composition further comprises methoprene. In another embodiment, the composition further comprises permethrin. More particularly, said animal is a dog, said ectoparasiticide is imidacloprid, and the composition further comprises permethrin. Alternatively, the ectoparasiticide is dinotefuran and the composition further comprises permethrin and pyriproxifen.

In another embodiment, the ectoparasiticide is applied to the animal in 1, 2, 3, 4, or 5 locations (spots), preferably down the back of the animal. More particularly, the ectoparasiticide is metaflumizone. Alternatively, the composition comprises metaflumizone and an additional ectoparasiticide, such as a pyrazole e.g. fipronil, pyriprole; a neonicotinoid e.g. imidacloprid, dinotefuran; a pyrethroid e.g. permethrin, pyrethrins; an insect growth regulator e.g. pyriproxyfen, S-methoprene or a mixture thereof. More particular still, the composition comprises 26%-40% metaflumizone. More particular still, the composition further comprises amitraz.

In another embodiment, the ectoparasiticide selectively kills fleas. In another embodiment the ectoparasiticide selectively kills fleas and ticks. In another embodiment, the composition comprises an ectoparasiticide and an additional insecticidal agent that does not kill fleas. Preferably, the additional agent selectively kills ticks.

In another embodiment, said animal is a dog. In another embodiment, said animal is a cat. In another embodiment, said animal is a farm animal. In another embodiment, said animal is a horse.

In another embodiment, said dose is greater than about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 42, 44, 45, 50, 55, 60, 65, 70, 75, 80, 85, 90, 95, 100, 105, 110, 115, 120, 125, 130, 135, 140, 145, 150, 160, 170, 180, 190, 200, 220, 240, 260, 280, 300 or 400 mg ectoparasiticide per kg of body weight.

In another embodiment, said dose is about: 20-200, 25-175, 25-150, 30-150, 30-130, 35-120, 35-100, 40-120, 40-100, 40-90, 40-80, 45-90, 45-80, 45-120, 45-180, 50-180, 50-160, 50-120, 50-100, 60-180, 60-150, 60-120, 60-100, 70-200, 70-150, 70-100, 80-150, 80-120, 80-100, 90-150, 90-120 or 90-100 mg per kg of body weight. In another embodiment, said dose is applied to a cat. In another embodiment, said dose is about 35-120 mg per kg of body weight and the animal is a dog. In another embodiment, said dose is about 35-100 mg per kg of body weight and the animal is a dog. In another embodiment said dose is about 40-80 mg metaflumizone per kg of body weight. In another embodiment, said dose is applied to a dog. In another embodiment the dose is applied to a cat.

In another embodiment, the composition is metaflumizone and said dose is about 35-100 mg per kg of body weight. In another embodiment, the composition is metaflumizone and said dose is about 40-80 mg metaflumizone per kg of body weight.

In another embodiment, said animal is a cat and said dose is about 60-240 mg per kg of body weight. More particularly, said dose is about 70-160 mg per kg of body weight. More particular still, said dose is about 80-160 mg per kg of body weight.

In another embodiment, said ectoparasiticide is fipronil and said dose is about 10-50 mg per kg of body weight. More particularly, said dose is about 15-45 mg per kg of body weight. More particular still, said dose is about 15-40 mg per kg of body weight. More particularly, said dose is about 15-35 mg per kg of body weight.

In another embodiment, said ectoparasiticide is imidacloprid and said dose is about 15-60 mg per kg of body weight. More particularly, said dose is about 20-50 mg per kg of body weight.

In another embodiment, said ectoparasiticide is pyriprole and said dose is about 30-200 mg per kg of body weight. More particularly, said dose is 35-100 mg per kg of body weight. More particularly, said dose is 35-75 mg per kg of body weight. In another embodiment the concentration of pyriprole in the composition is 13%-40% w/v, 15%-40% w/v, 20%-40% w/v, 25%-40% w/v or about 30%-40% w/v. In another embodiment the composition further comprises butylhydroxytoluene.

In another embodiment, said ectoparasiticide is dinotefuran and said dose is about 30-150 mg per kg of body weight. In another embodiment the concentration of dinotefuran in the composition is 5%-40% w/v, 6%-30% w/v, 7%-25% w/v, 8%-20% w/v or about 10%-20% w/v. In another embodiment the composition further comprises permethrin and/or pyriproxifen.

In another embodiment, the ectoparasiticide is present in a composition comprising about 5%-50% w/v, 5%-45% w/v, 10%-45% w/v, 15%-45% w/v, 20%-45% w/v, 25%-45% w/v, 5%-40% w/v, 10%-40% w/v, 15%-40% w/v, 20%-40% w/v, 25%-40% w/v, 30%-40% w/v, 5%-35% w/v, 10%-35% w/v, 15%-35% w/v, 20%-35% w/v, 25%-35% w/v, 30%-35% w/v, 5%-30% w/v, 10%-30% w/v, 15%-30% w/v, 20%-30% w/v, or 25%-30% w/v of said ectoparasiticide. Preferably, the ectoparasiticide is metaflumizone, dinotefuran, imidacloprid, or fipronil.

In another aspect of the invention, the ectoparasiticide is in a composition comprising:
(a) about 5% to about 40% of ectoparasiticide;
(b) about 5% to about 25% of a bridging agent;
(c) about 0% to about 15% of a surfactant; and
(d) about 5% to about 80% of a carrier solvent or a mixture of solvents.

Another aspect of the invention provides a composition comprising on a weight to volume basis:
(a) 26% to about 40% of metaflumizone;
(b) about 4% to about 25% of a bridging agent;
(c) about 0% to about 15% of a surfactant; and
(d) about 5% to about 70% of a carrier solvent or a mixture of solvents.

In another embodiment the metaflumizone is present from 27%-40% w/v, 28%-40% w/v, 29%-40% w/v, 30%-40% w/v, 27%-35% w/v, 28%-35% w/v, 29%-35% w/v or 30%-35% w/v. In another embodiment the metaflumizone is present at about 30% w/v. In another embodiment the metaflumizone is present at 30% w/v.

More particularly, the ectoparasiticide is metaflumizone, fipronil or imidacloprid. More particularly, the ectoparasiticide is metaflumizone and present at 26% to 35% on a weight to volume basis.

In another embodiment, said bridging agent is present at about 5% to about 15% w/v.

In another embodiment, said bridging agent is selected from the group consisting of an alkyl methylsulfoxide, dimethylsulfoxide (DMSO), decylmethyl sulfoxide, tetradecylmethyl, sulfoxide, a pyrrolidone, 2-pyrrolidone, N-methyl-2-pyrrolidone, N-(2-hydroxyethyl)pyrrolidone, a laurocapram, a solvent, acetone, dimethyl acetamide, dimethylformamide, and tetrahydrofurfuryl alcohol. In another embodiment, the bridging agent is selected from the group consisting of an L-amino acid, dimethylsulfoxide (DMSO) and a fatty acid.

In another embodiment, the surfactant is selected from the group consisting of an alcohol alkoxylate surfactant, a nonylphenol ethoxylate, an anionic or cationic surfactant, and a non-ionic surfactant.

In another embodiment, the carrier solvent is selected from the group consisting of a diluent, an adjuvant, an excipient, a preservative, and a vehicle with which a compound or composition is administered. In another embodiment, said carrier solvent is selected from the group consisting of petroleum oil, animal oil, vegetable oil, peanut oil, soybean oil, mineral oil, and sesame oil. Preferably, said carrier solvent comprises γ-hexalactone.

In another embodiment, the composition further comprises a second carrier solvent selected from the group consisting of N,N-diethyl-m-toluamide, eucalyptol, dimethyl isosorbide, diisopropyl adipate, and 1-methoxy-2-propyl acetate.

In another embodiment, the composition does not comprise a surfactant.

In a particular embodiment, the composition comprises, on a weight to volume basis, between about 15% and about 35% ectoparasiticide; about 10% of the bridging agent dimethyl sulfoxide; and between about 45% and about 60% of the carrier solvent γ-hexalactone.

In a particular embodiment, the composition comprises, on a weight to volume basis, between about 26% and about 35% ectoparasiticide; about 10% of the bridging agent dimethyl sulfoxide; and between about 45% and about 60% of the carrier solvent γ-hexalactone.

In a particular embodiment, the composition comprises, on a weight to volume basis, between about 25% and about 35% metaflumizone; about 10% of the bridging agent dimethyl sulfoxide; and between about 45% and about 60% of the carrier solvent γ-hexalactone.

In another embodiment, the composition further comprises a preservative selected from the group consisting of methylparaben, propylparaben, thiomersal, and EDTA.

In another embodiment, the composition further comprises a gelling agent selected from the group consisting of colloidal silicone dioxide, ethyl cellulose, methyl cellulose, a methacrylic ester copolymer, a carboxylated vinyl acetate terpolymer, a polyvinylpropylene (PVP)/Vinyl acetate copolymer, polyvinylmethylether, poly(vinylmethylether/maleic anhydride, an ethyl or butyl ester of polyvinylmethylether/maleic anhydride copolymer, and an ethyl or butyl ester of a PVM/MA copolymer.

In another embodiment, the ectoparasiticide is present in a concentration of 10-450 mg/mL, 50-750 mg/mL, 100-500 mg/mL, 150-400 mg/mL, or 200-350 mg/mL. In another embodiment, the ectoparasiticide is present at above 50 mg/mL, 60 mg/mL, 70 mg/mL, 80 mg/mL, 90 mg/mL, 100 mg/mL, 110, mg/mL, 120, mg/mL, 130 mg/mL, 140 mg/mL, 150 mg/mL, 160 mg/mL, 170 mg/mL, 180 mg/mL, 190 mg/mL, 200 mg/mL, 210 mg/mL, 220 mg/mL, 230 mg/mL, 240 mg/mL, 250 mg/mL, 260 mg/mL, 270 mg/mL, 280 mg/mL, 290 mg/mL, 300 mg/mL, 350 mg/mL, 400 mg/mL or 450 mg/mL.

In another embodiment, the total volume of the composition is less than 15 mL, 14 mL, 13.5 mL, 13 mL, 12.5 mL, 12 mL, 11.5 mL, 11 mL, 10.5 mL, 10 mL, 9.5 mL, 9 mL, 8.5 mL, 8 mL, 7.5 mL, 7 mL, 6.5 mL, 6 mL, 5.5 mL, 5 mL, 4.5 mL, 4 mL, 3.5 mL, 3 mL, 2.5 mL, 2 mL, 1.5 mL, 1 mL or 0.5 mL.

In another embodiment the animal is a dog, the ectoparasiticide is metaflumizone and the respective dose, animal body wt, volume, and concentrations are as shown in Table 13. In another embodiment, the animal is a cat, the ectoparasiticide is metaflumizone and the dose, animal body wt, volume, and concentrations are as shown in Table 14.

Another aspect of the invention provides a kit for preventing or treating an ectoparasite infestation in a warm blooded animal comprising a topical unit dose formulation of an ectoparasiticide comprising on a weight by volume basis:
(a) about 5% to about 40% of an ectoparasiticide;
(b) about 5% to about 25% of a bridging agent;
(c) about 0% to about 15% of a surfactant; and
(d) about 5% to about 80% of a carrier solvent or a mixture of solvents;
   wherein the unit dose comprises about 1.5 to 6 times the amount of the ectoparasiticide as compared to a conventional dose of said ectoparasiticide.

Another aspect of the invention provides a kit for preventing or treating an ectoparasite infestation in a dog or cat comprising:
a composition comprising on a weight to volume basis:
   (a) about 15% to about 40% of an metaflumizone;
   (b) about 5% to about 25% of a bridging agent;
   (c) about 0% to about 15% of a surfactant; and
   (d) about 5% to about 80% of a carrier solvent or a mixture of solvents; and
instructions for topical administration of the composition to the dog or cat.

More particularly, the ectoparasiticide is metaflumizone, fipronil or imidacloprid.

In a more particular embodiment, the kit comprises a unit dose formulation of the composition for administration to the dog or cat in an amount comprising 15, 16, 17, 18, 19, 20, 21, 22, 23, 24, 25, 26, 27, 28, 29, 30, 31, 32, 33, 34, 35, 36, 37, 38, 39, 40, 41, 42, 42, 44, 45, 50, 55, 60, 65, 70, 75, 80, 85, 90, 95, 100, 105, 110, 115, 120, 125, 130, 135, 140, 145, 150, 160, 170, 180, 190, 200, 220, 240, 260, 280, 300 or 400 mg/kg ectoparasiticide per kg of animal body weight. In another embodiment, said dose is about 35-120 mg per kg of body weight. In another embodiment, said dose is about 35-100 mg per kg of body weight. In another embodiment said dose is about 40-80 mg metaflumizone per kg of body weight. In another embodiment, said dose is about: 20-200, 25-175, 25-150, 30-150, 30-130, 35-120, 35-100, 40-120, 40-100, 40-90, 40-80, 45-90, 45-80, 45-120, 45-180, 50-180, 50-160, 50-120, 50-100, 60-180, 60-150, 60-120, 60-100, 70-200, 70-150, 70-100, 80-150, 80-120, 80-100, 90-150, 90-120 or 90-100 mg per kg of body weight.

In another embodiment, said ectoparasiticide is metaflumizone, said animal is a dog and said unit dose comprises between about 35 mg and about 120 mg metaflumizone per kg of body weight of the dog to which said composition is to be administered. In another embodiment, the formulation further comprises amitraz.

In another embodiment, said ectoparasiticide is metaflumizone, said animal is a cat and said single dose comprises between about 60 mg and about 240 mg metaflumizone per kg of body weight of the cat to which said composition is to be administered.

In another embodiment, said ectoparasiticide is imidacloprid, said animal is a cat or dog and said single dose comprises between about 15 mg and about 100 mg imidacloprid per kg of body weight of the cat or dog to which said composition is to be administered. More particularly, said animal is a dog and said formulation further comprises permethrin.

In another embodiment, said ectoparasiticide is fipronil, said animal is a cat or dog and said single dose comprises between about 10 mg and about 100 mg fipronil per kg of body weight of the cat or dog to which said composition is to be administered. More particularly, the animal is a dog and the formulation further comprises methoprene.

In another embodiment, said unit dose is effective in preventing or treating the ectoparasite infestation in said warm-blooded animal for a period greater than about 6 weeks.

As used in the specification and claims, the terms “about” and “approximately” designate that a value is within a statistically meaningful range. Such a range can be typically within 20%, more typically still within 10%, and even more typically within 5% of a given value or range. The allowable variation encompassed by the terms “about” and “approximately” depends on the particular system under study, and can be readily appreciated by one of ordinary skill in the art.

As used herein, the term “w/w” designates weight/weight, the term “w/v” designates weight/volume, and the term “mg/kg” designates milligrams per kilogram of body weight. The term “a.i.” or “ai” designates active ingredient, and may be combined with other terms. For example “mg a.i./kg” designates milligrams of active ingredient per kilogram of body weight.

The term “carrier” refers to a diluent, adjuvant, excipient, preservative, and/or vehicle with which a compound or composition is administered.

As used herein, the term “treating” or “treatment” of a condition, such as ectoparasite infestation includes inhibiting an existing condition or arresting its development; or ameliorating or causing regression of the condition. The term “preventing” or “prevention” of a condition, such as an ectoparasite infestation, includes substantially blocking or inhibiting the development or growth of a condition before it starts. Compositions that treat or prevent infestations herein will preferably exhibit at least 90% efficacy.

As used herein, the term “ectoparasiticide” refers to an agent that is capable of preventing, reducing or eliminating ectoparasite infestations. Preferred ectoparasiticides of the present invention include metaflumizone, fipronil, pyriprole, dinotefuran and imidacloprid.

As used herein, the term “conventional dose” refers to the dose which is disclosed and taught in the art for any one specific ectoparasiticide, i.e. the art-recognized dose, particularly the recommended dose provided by the manufacturer, such as the dose set forth on the label of a particular ectoparasiticide. The conventional dose of metaflumizone (ProMeris®) is 40 mg/kg for cats and 20 mg/kg for dogs, in a 20% w/v formulation in cats and 15% w/v formulation in dogs (with amitraz). The conventional dose of fipronil (Frontline®) is approximately 7 mg/kg in a 10% w/v formulation. The conventional dose of imidacloprid (Advantage®) is approximately 10 mg/kg in a 10% w/v formulation. The conventional dose for pyriprole (Prac-Tic®) is 12-28 mg/kg body weight and in a formulation of 12.5% w/v of pyriprole. The conventional dose for dinotefuran (Vectra3D™) is 7-16 mg/kg body weight and in a formulation of 5% w/v of dinotefuran.

High-dose, long-acting metaflumizone compositions as used herein comprise about 20% to 40% metaflumizone, preferably 26% to 40% metaflumizone on a weight to volume basis. Most typically, compositions comprise metaflumizone at a concentration selected from the group consisting of: 20%; 21%; 22%; 23%; 24%; 25%; 26%; 27%; 28%; 29%; 30%; 31%; 32%; 33%; 34%; 35%; 36%; 37%; 38%; 39%; and 40% on a weight to volume basis.

Metaflumizone is known in the art by its chemical name: (E,Z)-2-[2-(4-cyanophenyl)-1-[3-(trifluoromethyl)phenyl]ethylidene]-N-[4-(tri-fluoromethoxy)phenyl]hydrazinecarboxamide and is described in U.S. Pat. No. 5,543,573, and U.S. 2004/0122075A1, among other publications. The chemical structure of metaflumizone is shown below.

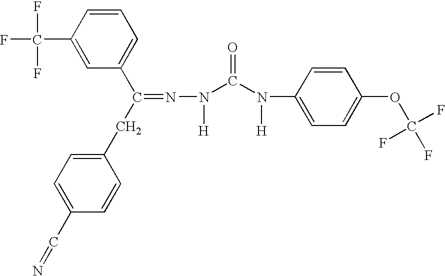

ProMeris® is the trade name for the veterinary formulation of metaflumizone marketed by Fort Dodge Animal Health (Wyeth). ProMeris® Spot-On for Cats is a product containing metaflumizone in a non-aqueous solution, designed as a topically applied treatment to control fleas on cats at a rate of 40 mg/kg body weight. ProMeris® Spot-On for Dogs is a product containing metaflumizone and amitraz in a non-aqueous solution, designed as a topically applied treatment to control fleas and ticks on dogs at a rate of 20 mg/kg body weight of each active ingredient.

High-dose, long-acting compositions of the present disclosure may alternatively or additionally comprise one or more alternative ectoparasiticide agents such as, for example, imidacloprid, fipronil, amitraz, or a mixture thereof.

Frontline® is the trade name for the veterinary formulation of fipronil marketed by Merial Limited (Duluth, Ga.). Frontline® is a non-aqueous solution, designed as a topically applied treatment to control fleas and ticks with a rate of 7 mg/kg body weight in a formulation of 10% w/v of fipronil. Frontline® Plus further comprises the active ingredient methoprene. The chemical structure of fipronil is shown below:

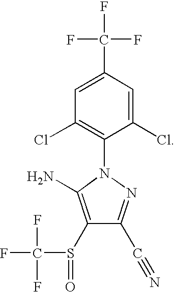

Advantage® and Advantix® are trade names for veterinary formulations of imidacloprid marketed by Bayer Corporation (Shawnee Mission, Kans.). Advantage® is a non-aqueous solution, designed as a topically applied treatment to control fleas with a rate of 10 mg/kg and in a formulation of 10% w/v of imidacloprid. Advantix® for use on dogs, further comprises the active ingredient permethrin. The chemical structure of imidacloprid is shown below:

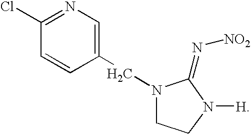

Prac-tic® is the trade name for veterinary formulation of pyriprole marketed by Novartis Corp. Prac-tic® is a non-aqueous solution, designed as a topically applied treatment to control fleas with a rate of 12-28 mg/kg body weight and in a formulation of 12.5% w/v of pyriprole. The chemical structure of pyriprole is shown below:

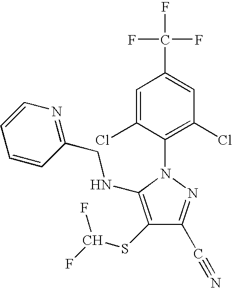

Vectra3D™ is the trade name for veterinary formulation of dinotefuran marketed by Summit Vet Pharm. Vectra3D™ is a solution, designed as a topically applied treatment to control fleas with a rate of 7-16 mg/kg body weight and in a formulation of 5% w/v of dinotefuran. Vectra3D™ also contains permethrin and pyriproxifen. The chemical structure of dinotefuran is shown below:

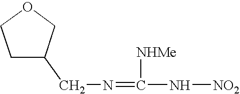

In certain embodiments about 5% to 15% on a weight to volume basis of a bridging agent is included in the compositions disclosed herein. In other embodiments about 5% to about 25% on a weight to volume basis of a bridging agent is included in the compositions disclosed herein. Most typically, compositions comprise one or more of the bridging agent at a concentration selected from the group consisting of: 5%; 6%; 7%; 8%; 9%; 10%; 11%; 12%; 13%; 14%; and 15% on a weight to volume basis.

Bridging agents that are suitably employed in the compositions disclosed herein include, without limitation, alkyl methyl sulfoxides (such as dimethylsulfoxide (DMSO), decylmethyl sulfoxide and tetradecylmethyl sulfoxide); pyrrolidones (such as 2-pyrrolidone, N-methyl-2-pyrrolidone and N-(2-hydroxyethyl)pyrrolidone); laurocapram; and miscellaneous solvents such as acetone, dimethyl acetamide, dimethyl formamide, and tetrahydrofurfuryl alcohol. Other bridging agents include amphiphiles such as L-amino acids, and fatty acids. Additional bridging agents are disclosed in Remington: The Science and Practice of Pharmacy, 19th Edition (1995), on page 1583.

The composition of the invention additionally comprises about 0% to 15% of a surfactant on a weight to volume basis. Most typically, the surfactant is present at a concentration selected from the group consisting of: 0%; 1%; 2%; 3%; 4%; 5%; 6%; 7%; 8%; 9%; 10%; 11%; 12%; 13%; 14%; and 15% on a weight to volume basis.

Surfactants suitably employed in the composition of the invention include a single surfactant, or a mixture of two or more surfactants. Suitable surfactants are non-irritating and non-toxic. Exemplified herein, without limitation, are non-ionic, low foaming surfactants, such as the alcohol alkoxylate surfactants sold by Uniqema under the tradename Synperonic® NCA 810, 830 and 850. Other suitable surfactants are the nonylphenol ethoxylates, such as those sold under the tradename Tergitol®NP by the Dow Chemical Company. Additional surfactants, including appropriately chosen anionic and cationic surfactants, can also be utilized in the composition of the present invention. Especially useful properties are found in anionic surfactants, such as dioctylsulfosuccinate salts. Particularly effective surfactants for use with an organic carrier solvent are non-ionic surfactants such as polyoxyl 35 castor oil sold under the Cremophor® trade name.

The inventive composition may also comprise one or more carrier solvents which may be present at about 5% to 80% on a weight to volume basis. Most typically, the carrier solvent or mixture of solvents is present at a concentration selected from the group consisting of: about 5% to 45%; about 50%; about 55%; about 60%; about 65%; about 70%; about 75%; and about 80% on a weight to volume basis.

Carrier solvents that may be suitably employed in the composition of the invention include single solvents, or a mixture of two or more solvents. Due to the instability of metaflumizone in the presence of primary alcohols, preferred solvents are non-hydroxyl-group-containing solvents, especially those such as γ-hexalactone (also known as γ-caprolactone; ethyl butyrolactone; γ-ethyl-n-butyrolactone; hexanolide-1,4; 4-hydroxy hexanoic acid γ-lactone or tonkalide), δ-hexalactone, γ-butyrolactone etc. γ-Hexalactone, Synperonic NCA 830, and dimethyl sulfoxide are employed within some embodiments of the present invention. Within other embodiments, solvents such as N,N-diethyl-m-toluamide, eucalyptol, dimethyl isosorbide, diisopropyl adipate and/or 1-methoxy-2-propyl acetate may be advantageously utilized in combination with the γ-hexalactone to comprise the carrier solvent mixture. Suitable pharmaceutical carriers are described in “Remington's Pharmaceutical Sciences” by E. W. Martin, 18^{th} Edition. A wide variety of carriers are readily available and the selection of specific carriers is well within the level of those skilled in the art.

To manufacture a high-dose, long-acting metaflumizone composition of the present invention, the metaflumizone may be dissolved in the carrier solvent or solvents and bridging agent, and the surfactant, if desired, added to the metaflumizone/carrier solvent solution. These compositions may then be utilized as a high-dose, long-acting spot-on for topical application or may be further diluted for alternative uses. Compositions are most suitably formulated as creams, gels, solutions, or in microspheres.

An exemplary composition for topical administration to warm-blooded animals typically comprises, on a weight to volume basis, about 5%-40% w/v metaflumizone; about 10% w/v of a bridging agent, such as dimethyl sulfoxide; about 0%-8% w/v of a non-ionic, low foam surfactant; and about 45%-60% of a carrier solvent or solvent mixture, such as γ-hexalactone by itself or in combination with about 10% w/v of N,N-diethyl-m-toluamide, about 10% w/v of eucalyptol and about 20% w/v of 1-methoxy-2-propyl acetate.

High-dose, long-acting metaflumizone compositions of the invention may further comprise other agents known in the art, such as preservatives (e.g., methylparaben and propylparaben), colorants, antioxidants, or the like. Generally, these agents are present in the composition in an amount up to about 2% on a weight to volume basis. Preservatives may include, for example, thiomersal, EDTA, or the like.

Suitable exemplary polymers (“polymeric agents”) for gelling and/or adhering that may be used in the compositions of the invention include, but are not limited to, colloidal silicone dioxide, ethyl cellulose, methyl cellulose, methacrylic esters copolymers, carboxylated vinyl acetate terpolymer, Resyn® 29-2930, and polyvinylpropylene (PVP)/Vinyl acetate copolymers. “Gantrez”® is the trade name for a family of polyvinylmethylether formulations (as solutions, creams, powders, etc.) manufactured by International Specialty Products, of Wayne, N.J. Gantrez powder formulations comprise poly(vinylmethylether/maleic anhydride). Gantrez cream formulations comprise ethyl or butyl esters of polyvinylmethylether/maleic anhydride copolymer and ethyl or butyl esters of PVM/MA copolymer. Resyn® 29-2930 is the trade name for vinyl acetate/crotanoates/vinylneodecanoate copolymer.

Carbopol® is the trade name of exemplary carbomers used in the formulation of certain embodiments of the presently disclosed compositions. Other acrylic acid polymers other than carbomers may be used, though carbomers may be employed.

In those embodiments in which high-dose, long-acting metaflumizone compositions are formulated as microspheres, polymers including, but not limited to, sulfopolyester (AQ55S polymer from Eastman Chemical Co., Kingsport, Tenn., USA; typical repeating units of these polymers are disclosed in column 7 of U.S. Pat. No. 5,260,052.), and cellulose acetate butyrate (CAB), poly(lactide-co-glycolide) (PLGA) may be employed. Alternatively, microsphere formulation may employ one or more of polylactic galactide, hollow microspheres such as Expancel (Nobel Industrie), Polytrap (Dow Corning), and hollow silica microspheres (Silica Beads from Maprecos).

Additionally, UV-absorbing compounds, photostabilizers, viscosity modifying agents, antimicrobial agents, dyes, thickeners, antioxidants, taste enhancers or deterrents, vitamins, adherents, perfumes, deodorants, physiologically or dermatologically acceptable carriers, diluents, excipients or adjuvants may be included in the compositions and formulations of the present invention.

Advantageously, the ectoparasiticidal topical veterinary composition of the invention allows for high concentrations of the active ingredients and demonstrates no irritation to the skin/hide/hair of the host animal. Accordingly, the present invention provides a method for the treatment of an ectoparasiticidal infection or infestation in a warm-blooded animal which comprises topically administering to said animal a composition which comprises an effective amount of metaflumizone; a bridging agent or penetration enhancer, an optional surfactant, and a carrier solvent or mixture of solvents.

When topically administered, the high-dose, long-acting ectoparasiticidal composition of the invention is highly effective for preventing or mitigating ectoparasitic infection and/or infestation for prolonged periods of time in warm-blooded animals such as swine, cattle, sheep, horses, goats, camels, water buffalo, bison, donkeys, rabbits, kangaroos, fallow deer, reindeer, minks, chinchillas, raccoons, chicken, geese, turkeys, ducks, dogs, cats, or the like, preferably dogs, cats, swine, cattle, horses or sheep, and most preferably cats or dogs.

Examples of topical administrations suitable for use in the method of the invention include spot-on, pour-on, dip, wash, shampoo, foam, gel, lotion, cream, microsphere-encapsulated formulation, powder, or any of the conventional means of topically applying a liquid or semi-liquid veterinary composition. The topical mode of administration will vary with the species and size of the host animal. As an example, for companion animals such as dogs or cats, a spot-on, gel, cream, powder, or microsphere-encapsulated formulation, and most preferably a spot-on, may be suitable. For large agronomic animals such as cattle, horses or sheep, a pour-on or spray, most preferably a pour-on, may be suitable.

Ectoparasitic infection or infestations suitable for treatment by the methods of the invention include fleas, ticks, lice, mites and flies. In particular, the methods and kits of the present invention are suitable for treating or preventing flea infestations, and even more particularly, infestations of the cat flea, Ctenocephalides felis and the dog flea, Ctenocephalides canis.

In actual practice, the composition of the invention may be administered in dose rates of mg of active ingredient per kg of body weight of the host animal. As will be understood by those skilled in the art, dose rates suitable for use in the method of invention will vary depending upon the identity of the ectoparasiticide, the mode of administration, the species and health of the host animal, the target parasite, the degree of infection or infestation, the breeding habitat, the potency of the additional parasiticidal compound, or the like.

For dogs, a dose of metaflumizone of about 30-120 mg per kg of body weight may be administered. More typically, a dose of metaflumizone will be about 35-100 mg per kg of body weight or about 40-80 mg metaflumizone per kg of body weight.

For cats, a dose of metaflumizone of about 60-240 mg per kg of body weight may be administered. More typically, a dose of metaflumizone will be about 70-160 mg per kg of body weight or about 80-160 mg metaflumizone per kg of body weight.

Typical “spot-on” applications of exemplary embodiments of the invention are applied to the base of the neck of the animal or generally along the dorsal midline in the area between the shoulder blades, so as to aid in making the applied composition difficult for the animal to remove.

Surprisingly, it has now been found that dosages of two to four times the conventional dose rates of an ectoparasiticide in topical formulations offer substantially longer effective protection against ectoparasites in warm-blooded animals, including both dogs and cats. Accordingly the present invention provides a method for extending the period of efficacy of an ectoparasiticide which comprises administering a high dose of said ectoparasiticide. High doses suitable for use in the method of the invention include dose rates of about 2 to 4 times the conventional dose rate for said ectoparasiticide. Beneficially, the method of the invention provides protection from ectoparasitic infestation for an extended period of time compared to presently available commercial formulations. The extended period of efficacy obtained by the method of the invention may be greater than about 6 weeks and may be about 6-20 weeks of extended efficacy.

In one embodiment of the invention, for dogs or cats, a dose of imidacloprid of about 15-60 mg per kg of body weight may be administered. More typically, a dose of imidacloprid of about 20-50 mg per kg of body weight may be administered to obtain extended efficacy against ectoparasites of about six week or greater.

In another embodiment of the invention, for dogs or cats, a dose of fipronil of about 10-50 mg per kg of body weight may be administered. More typically, a dose of fipronil of about 15-35 mg per kg of body weight may be administered to obtain protection from ectoparasitic infestation for an extended period of time compared to presently available commercial formulations.

Topical administration of a high dose, such as 2-4 times the conventional dose, of compositions and formulations containing ectoparasiticides such as metaflumizone, fipronil, imidacloprid or the like, or a mixture thereof are suitable for use in the method of the invention for the long-term protection of warm-blooded animals against ectoparasital infection or infestation. Advantageously, the method of the invention increases the period of time for effectively reducing or controlling the proliferation of acarid or arthropod ectoparasites.

For a more clear understanding of the invention, the following examples are set forth hereinbelow. These examples are merely illustrative and are not understood to limit the scope or underlying principles of the invention in any way. Indeed, various modifications of the invention, in addition to those shown and described herein, will become apparent to those skilled in the art from the examples set forth hereinbelow and the foregoing description. Such modifications are also intended to fall within the scope of the appended claims.

Unless otherwise designated, all parts are parts by weight/volume. The term qs designates quantity sufficient to obtain a total of 100%. The term DMSO designates dimethyl sulfoxide. The term G.M. designates geometric mean.

### EXAMPLE 1

### Preparation of a Metaflumizone High-Dose, Long-Acting Concentrate Composition, Suitable for Use as a Spot-On Treatment

| | A | B | C |
|---|---|---|---|
| Ingredients | (% w/v) | (% w/v) | (% w/v) |
| Metaflumizone | 30 | 30 | 20 |
| Synperonic NCA 830 | 5 | 5 | 5 |
| DMSO | 10 | 10 | 10 |
| Resyn ® 29-2930 | — | 5 | — |
| γ-hexalactone | qs | qs | qs |

### Composition A

To 10 grams of DMSO was added 40 grams of γ-hexalactone. To this solvent mixture was added 30 g of metaflumizone. Mild heat (40° C.) was used to facilitate the process of dissolution. To the resulting solution, 5 grams of Synperonic® NCA 830 brand of alcohol alkoxylate surfactant was added with stirring. This solution was brought to a final volume of 100 ml with γ-hexalactone.

### Composition B

To 10 grams of DMSO is added 40 grams of γ-hexalactone. To this solvent mixture is added 30 g of metaflumizone. Mild heat (40° C.) is used to facilitate the process of dissolution. To the resulting solution, 5 grams of Synperonic® NCA 830 brand of alcohol alkoxylate surfactant is added with stirring. To this is added 5 g of Resyn® 28-2930 brand of polymer and the mixture is stirred to dissolve the solids. This solution is brought to a final volume of 100 ml with γ-hexalactone.

### Composition C

Using essentially the same procedure described hereinabove for composition A, composition C was prepared.

Compositions A, B and C are applied as a “spot-on” treatment for cats or dogs.

### EXAMPLE 2

### Preparation of a Metaflumizone High-Dose, Long-Acting Concentrate Composition, Suitable for Use as a Spot-On Treatment

| Ingredients | (% w/v) |
|---|---|
| Metaflumizone | 30 |
| DMSO | 10 |
| N,N-Diethyl-m-toluamide | 10 |
| Eucalyptol | 10 |
| 1-Methoxy-2-propyl acetate | 20 |
| γ-hexalactone | qs |

### Composition D

To 10 grams of DMSO were added 10 grams of N,N-diethyl-m-toluamide, 10 grams of eucalyptol, 20 grams of 1-methoxy-2-propyl acetate and 20 grams of γ-hexalactone. To this solvent mixture was added 30 g of metaflumizone. The resultant mixture was stirred by mechanical means to facilitate dissolution. This resulting solution was brought to a final volume of 100 ml with γ-hexalactone.

This composition was applied as a “spot-on” treatment for cats or dogs.

Compositions E and F are prepared as described above, with the following mixtures on a weight by volume basis.

COMPOSITION E

| | Quantity | Quantity | Quantity | Quantity |
|---|---|---|---|---|
| Ingredients | (% w/v) | (% w/v) | (% w/v) | (% w/v) |
| Metaflumizone | 30.0 | 30.0 | 30.0 | 30.0 |
| Dimethyl sulfoxide | 10.0 | 10.0 | 10.0 | 10.0 |
| Synperonic ™ (grade | 5.0 | 5.0 | 5.0 | 5.0 |
| NCA 830) | | | | |
| Diethylene glycol mono- | 10.0 | — | — | — |
| ethyl ether | | | | |
| Polyvinyl pyrrolidone | — | 1.0 | — | — |
| (grade C30) | | | | |
| Phosal ® (grade 50PG) | — | — | — | 5.0 |
| Dermacryl ® (grade 79) | — | — | 1.0 | — |
| γ-Hexalactone | Qs ad | Qs ad | Qs ad | Qs ad |
| | 100 | 100 | 100 | 100 |

Synperonic NCA 830 is an ethoxylated fatty alcohol
Dermacryl is an acrylate/octylacrylamide copolymer
Phosal is a dispersion of 50% phosphatidylcholine in a propylene glycol/ethanol carrier

COMPOSITION F

| | Quantity | Quantity |
|---|---|---|
| Ingredients | (% w/v) | (% w/v) |
| Metaflumizone | 30.0 | 30.0 |
| Dimethyl sulfoxide | 11.6 | 11.6 |
| 1-Methoxy-2-propyl acetate | 23.0 | — |
| N,N-Diethyl-m-toluamide | 11.6 | 11.6 |
| Diethylene glycol monoethyl ether | — | 23.0 |
| Butylated hydroxytoluene | — | 0.20 |
| γ-Hexalactone | qs ad 100 | qs ad 100 |

### EXAMPLE 3

### Evaluation of the Efficacy of Test Compositions Against the Cat Flea, Ctenocephalides felis, in Cats

In this evaluation, domestic shorthair cats of mixed sex, aged approximately 4 years, were used. Cats were free of ectoparasiticide treatment for 60 days prior to administration of the Test Compositions. No drugs, baths, shampoos, or pesticides were given to the cats during the preconditioning phase or during the course of the study other than what is described in the protocol. The cats were preconditioned for 13 days. On day −13, each cat was infested with 100 unfed cat fleas, Ctenocephalides felis. On Day −12, each cat was thoroughly examined and combed to remove and count fleas. The cats were ranked in descending order by flea count. From this ranking, cats were blocked into three groups. The cats within each of the three blocks were randomly allocated to treatment groups (A-G). The cats were weighed on Day −1 and received one of the treatments presented in Table 1.

TABLE 1
FLEA TREATMENTS APPLIED TO CATS

| | | | Appli- | |
|---|---|---|---|---|
| | | | cation | |
| | Number | | Rate, | Dose mg |
| Group | of cats | Treatment¹ | ml/kg | ai/kg BW |
| A | 3 | Untreated | 0 | 0 |
| B | 3 | 30% w/v Metaflumizone, | 0.27 | 80 |
| | | 5% polymer | | |
| C | 3 | 30% w/v Metaflumizone, | 0.27 | 80 |
| | | 1% polymer | | |
| D | 3 | 20% w/v Metaflumizone, | 0.2 | 40 |
| | | 5% polymer | | |
| E | 3 | 20% w/v Metaflumizone, | 0.2 | 40 |
| | | 1% polymer | | |
| F | 3 | 30% w/v Metaflumizone, | 0.27 | 80 |
| | | 0% polymer | | |
| G | 3 | 20% w/v ProMeris ® Spot on | 0.2 | 40 |

¹The “polymer” used in the treatments listed in Table 1 was Resyn 28-2930, available from National Starch and Chemical. Other excipients used in the above formulations were Synperonic NCA830, gamma hexalactone GMP04-15, and DMSO, Sigma-Aldrich.

Applications for groups B-G were administered using a disposable pipette. The dose was applied as a single spot on the dorsal neck at the base of the skull. Group A was left untreated. The cats were observed for any immediate reactions to the treatments, and were observed for post-treatment adverse reactions, skin irritation, and behavior of test formulations at the time of treatment, after approximately four hours, and on Days 1 and 2 following administration of the treatments. Thereafter, cats were observed once daily for the remainder of the study.

Cats were infested with 100 adult cat fleas on Day −1, then examined for live fleas on Day 2 to determine knockdown. The cats were reinfested on Day 13 then examined for fleas on Day 15. The cats were reinfested and examined yet again on Days 27 and 29, then monthly until efficacy waned.

At each infestation, 100 unfed adult fleas were applied along the dorsal rump of each cat. The negative control group (Group A) was infested before the test compound groups. Fleas were recovered by examining and combing the groups in the same order. Combs were washed and rinsed with alcohol between cats, and latex gloves were changed between treatment groups.

Flea counts were transformed by log (count +1), and geometric means were used to calculate percent efficacy for the treatments using (Yc−Yt)/Yc*100, where Yc and Yt equal mean counts for control and treated groups, respectively.

The results are shown in Table 2 hereinbelow. As shown in Table 2, of the formulations tested in this study, the high-dosage metaflumizone formulation with no polymer (Group F) offered the best long-term protection against fleas—providing very high efficacy (94.1%) at Day 85, and higher efficacy at Day 113 (74.9%) than most other formulations exhibited at Day 85.

TABLE 2
AVERAGE FLEA COMB COUNTS FOR CATS ADMINISTERED
VARIOUS TEST COMPOSITIONS

| Tr. Group¹ | | Day −12 | Day 2 | Day 15 | Day 29 | Day 57 | Day 85 | Day 113 |
|---|---|---|---|---|---|---|---|---|
| A | G.M.² | 52.5 | 53.9 | 83.9 | 62.9 | 65.2 | 74.0 | 66.9 |
| B | G.M. | 55.8 | 1.9 | 0.7 | 0.0 | 10.1 | 27.7 | NT³ |
| | % Efficacy | | 96.5 | 99.2 | 100.0 | 84.5 | 62.5 | NT |
| C | G.M. | 51.2 | 0.8 | 0.3 | 0.8 | 2.6 | 15.9 | NT |
| | % Efficacy | | 98.5 | 99.7 | 98.7 | 96.1 | 78.5 | NT |
| D | G.M. | 53.9 | 10.5 | 0.6 | 5.5 | 23.3 | 44.3 | NT |
| | % Efficacy | | 80.4 | 99.3 | 91.3 | 64.3 | 40.2 | NT |
| E | G.M. | 59.9 | 0.3 | 0.0 | 1.5 | 4.1 | 36.4 | NT |
| | % Efficacy | | 99.5 | 100.0 | 97.6 | 93.7 | 50.9 | NT |
| F | G.M. | 55.1 | 0.6 | 0.3 | 0.0 | 0.36 | 4.3 | 16.8 |
| | % Efficacy | | 98.9 | 99.7 | 100.00 | 99.1 | 94.1 | 74.9 |
| G | G.M. | 58.8 | 0.0 | 0.0 | 0.0 | 1.9 | 14.8 | NT |
| | % Efficacy | | 100.0 | 100.0 | 100.0 | 97.1 | 80.0 | NT |

¹Treatments:
A = untreated;
B = 30% w/v metaflumizone, 5% polymer, at 80 mg/kg
C = 30% w/v Metaflumizone, 1% polymer, at 80 mg/kg
D = 20% w/v Metaflumizone, 5% polymer, at 40 mg/kg
E = 20% w/v Metaflumizone, 1% polymer, at 40 mg/kg
F = 30% w/v Metaflumizone, 0% polymer, at 80 mg/kg
G = 20% w/v ProMeris ® Spot-on, at 40 mg/kg
²G.M. = Geometric mean
³NT = not tested

These data demonstrate the surprising long-term efficacy of novel high-dosage metaflumizone formulations in the treatment of cat fleas in cats.

### EXAMPLE 4

### Evaluation of the Efficacy of Test Compositions Against the Cat Flea, Ctenocephalides felis, in Dogs

In this evaluation, Beagles weighing not more than 10 kg, aged one to 10 years, were used. Dogs were free of ectoparasiticide treatment for 60 days prior to administration of the long-acting formulations. No drugs, baths, shampoos or pesticides were given to the dogs during the preconditioning phase or during the course of the study other than what is described in the protocol. The dogs were preconditioned for 14 days. All dogs were bathed with a noninsecticidal shampoo between Days −12 to −10. On Day −7, each dog was infested with 100 unfed cat fleas, Ctenocephalides felis. On Day −6, each dog was thoroughly examined and then combed to remove and count fleas. Dogs were selected for inclusion on the basis of flea retention and/or behavior. The dogs with the highest flea counts were selected for the study. Dogs retained at least 30 fleas to be included in the present study. The animals were blocked by flea number retained into three blocks. Within blocks, the dogs were randomly allocated into groups (A-J) of 3 dogs each. The selected dogs were weighed on Day −1 and received flea treatments on Day 0. The five treatment groups included in this example are presented in Table 3, wherein the column headed n is the number of animals.

TABLE 3
FLEA TREATMENTS APPLIED TO DOGS

| | | | Application | Dose |
|---|---|---|---|---|
| Group | n | Treatment | Rate | (mg/kg) |
| A | 3 | Control - No treatment | 0 | 0 |
| B | 3 | 30% w/v metaflumizone spot-on | 0.2 ml/kg | 60 |
| H | 3 | 30% w/v metaflumizone/Gantrez | 0.2 ml/kg | 60 |
| I | 3 | 6% w/v metaflumizone/Gantrez | 1.0 ml/kg | 60 |
| | | complex in a suspension | | |
| J | 3 | 30% w/v metaflumizone cat spot- | 0.2 ml/kg | 60 |
| | | on with 5% polymer | | |

Groups B and J received a spot to the skin (by syringe) at the dorsal midline between the shoulder blades. The metaflumizone/Gantrez powder (Group H) and metaflumizone/Gantrez complex suspension (Group I) were applied (poured or shaken from an appropriate container) directly to the skin along the back and rubbed onto the skin using a gloved finger. Group A was left untreated. All metaflumizone treatments were dosed at 60 mg/kg. The dogs were observed for any immediate reactions to the treatments. Observations of the behavior and characteristics of the formulations were noted, with observations for post-treatment adverse reactions, skin irritation and behavior of test formulations made at approximately 4 and 24 hours following the administration of treatments. Thereafter, dogs were observed once daily on Days 2-14 for formulation acceptability, adverse reactions or signs of skin irritation following treatment. Daily observations of the physical condition of the dogs were made throughout the trial, in part to monitor for distress or injury.

Dogs were infested with 100 adult cat fleas on Day 14 and then examined for live fleas on Day 16. The dogs were reinfested on Day 28 and examined for fleas on Day 30, and then reinfested periodically for up to five months after treatment depending upon the flea control activity observed. At each infestation, 100 unfed adult fleas were applied along the lateral midline of each dog. The negative control group (Group A) was infested before the test compound groups. Fleas were recovered by examining and combing the groups in the same order. Combs were washed and rinsed with alcohol between dogs. Plastic gloves and aprons were changed between treatment groups.

Flea counts were transformed by log (count +1), and geometric means were used to calculate percent efficacy for the treatments using (Yc−Yt)/Yc*100, where Yc and Yt equal mean counts for control and treated groups, respectively. The results are shown in Table 4 hereinbelow, wherein the column headed n represents the number of animals.

As shown in Table 4, dogs treated with 30% metaflumizone spot-on formulation at 60 mg/kg (Group B) were almost completely protected (97.7% efficacy) from fleas at Day 114, and protected with 72.1% efficacy at Day 142. Overall, the spot-on formulations (Groups B and J) performed significantly better than other high-dose powder or suspension formulations tested (Groups H and I).

TABLE 4
AVERAGE FLEA COMB COUNTS FOR DOGS ADMINISTERED
VARIOUS TEST COMPOSITIONS

| | | Flea Counts/Dog (Geometric Means)¹ | | | | | | | |
|---|---|---|---|---|---|---|---|---|---|
| Group | | Day 16 | Day 30 | Day 58 | Day 86 | Day 100 | Day 114 | Day 128 | Day 142 |
| A | | 63.8 | 71.1 | 65.1 | 69.1 | 66.9 | 70.3 | 77.6 | 74.6 |
| B | % E² | 0.0 | 0.0 | 0.0 | 1.9 | 1.5 | 1.6 | 9.0 | 20.8 |
| | | 100.0 | 100.0 | 100.0 | 97.3 | 97.8 | 97.7 | 88.4 | 72.1 |
| H | % E² | 0.3 | 0.3 | 14.0 | 23.1 | NT | NT | NT | NT |
| | | 99.7 | 99.7 | 78.5 | 66.5 | | | | |
| I | % E² | 0.0 | 1.4 | 4.4 | 21.9 | NT | NT | NT | NT |
| | | 100.0 | 98.6 | 93.2 | 68.3 | | | | |
| J | % E² | 0.0 | 0.0 | 0.3 | 9.8 | 5.0 | 8.1 | NT | NT |
| | | 100.0 | 100.0 | 99.6 | 85.7 | 92.5 | 88.4 | | |

¹NT = Not tested.
²% Efficacy
Treatments:
A = untreated
B = 30% Metaflumizone Spot-On
H = 30% Metaflumizone/Gantrez Powder
I = 6% Metaflumizone/Gantrez Complex Suspension
J = 30% Metaflumizone Spot-On w/5% polymer

### EXAMPLE 5

### Evaluation of the Duration of Efficacy of High Doses (2× and 3× the Conventional Dose) of Ectoparasiticides Against the Cat Flea, Ctenocephalides felis, in Cats

In this evaluation, 32 domestic shorthair cats of mixed sex, aged at least 6 months, were used to test the high doses of ectoparasiticides disclosed herein. Cats were free of ectoparasiticide treatment for 60 days prior to administration of the experimental treatments. No drugs, baths, shampoos, or pesticides were given to the cats during the preconditioning phase or during the course of the study other than what is described in the protocol. At least 36 cats were preconditioned for 7 days. On day −7, each cat was infested with 100 unfed cat fleas, Ctenocephalides felis. On Day −5, each cat was thoroughly examined and combed to remove and count fleas. 32 cats were ranked in descending order by flea count. From the ranking, the cats were divided into four blocks of eight cats each. The eight cats within each of the four blocks were randomly allocated to treatment groups (A-H). The cats were weighed on Day −1 and received one of the treatments presented in Table 5.

TABLE 5
FLEA TREATMENTS APPLIED TO CATS

| | | | Application | |
|---|---|---|---|---|
| | Number | | Rate, | Dose |
| Group | of Cats | Treatment | ml/kg | mg/kg |
| A | 4 | Nontreated | 0 | 0 |
| B | 4 | 20% w/v Metaflumizone | 0.200 | 40 |
| C | 4 | 30% w/v Metaflumizone | 0.270 | 80 |
| D | 4 | 30% w/v Metaflumizone | 0.400 | 120 |
| E | 4 | 10% w/v fipronil* | 0.075 | 7.5 |
| F | 4 | 10% w/v fipronil* | 0.225 | 22.5 |
| G | 4 | 10% w/v imidacloprid** | 0.100 | 10 |
| H | 4 | 10% w/v imidacloprid** | 0.300 | 30 |

*The fipronil formulation used in the present Example was Frontline ®, as sold by Merial Limited (Duluth, GA)
**The imidacloprid formulation used in the present Example was Advantage ® as sold by Bayer Corporation (Shawnee Mission, KS)

Applications for groups B-H were administered using a disposable syringe. The dose was applied as a single spot on the dorsal neck at the base of the skull. Group A was left untreated. The cats were observed for any immediate reactions to the treatments, and were observed for post-treatment adverse reactions, skin irritation, and behavior of test formulations at the time of treatment, after approximately four hours. On Days 1 through 14 following administration of the treatments, cats were observed daily for reactions and formulation acceptability. Thereafter, cats were observed once daily for the remainder of the study. Cats were infested with 100 adult cat fleas on Day 14 then examined for fleas on Day 16. The cats were re-infested and examined again on Days 28 and 30, on Days 56 and 58 then bi-weekly until efficacy waned. At each infestation, 100 unfed adult fleas were applied along the dorsal rump of each cat. The negative control group (Group A) was infested before the test compound groups. Fleas were recovered by examining and combing the groups in the same order. Combs were washed and rinsed with alcohol between cats, and latex gloves were changed between treatment groups.

Flea counts were transformed by log (count +1), and geometric means were used to calculate percent efficacy for the treatments using (Yc−Yt)/Yc*100, where Yc and Yt equal mean counts for control and treated groups, respectively. The results are shown in Table 6 hereinbelow.

As shown in Table 6, cats treated with higher doses (2× or 3× the conventional dosage) of the three ectoparasiticides tested (metaflumizone, fipronil, and imidacloprid) experienced sustained protection from fleas for longer periods of time compared to cats treated with standard doses.

TABLE 6
AVERAGE FLEA COMB COUNTS FOR CATS ADMINISTERED HIGH-DOSE
RATES OF METAFLUMIZONE, FIPRONIL, OR IMIDACLOPRID

| Treatment Group | | Day 16 | Day 30 | Day 58 | Day 72 | Day 86 | Day 100 |
|---|---|---|---|---|---|---|---|
| A Untreated | G.M. | 52.5 | 63.4 | 54.1 | 66.3 | 57.9 | 59.0 |
| B | G.M. | 1.3 | 3.2 | 13.7 | 9.8 | 27.2 | 43.3 |
| 20% metaflumizone | % Efficacy | 97.4 | 94.9 | 74.7 | 85.2 | 53.1 | 26.5 |
| 40 mg/kg | | | | | | | |
| C | G.M. | 0.9 | 3.9 | 14.4 | 9.4 | 24.4 | 31.9 |
| 30% metaflumizone | % Efficacy | 98.3 | 93.9 | 73.4 | 85.8 | 57.8 | 45.9 |
| 80 mg/kg | | | | | | | |
| D | G.M. | 0.0 | 0.7 | 0.3 | 1.1 | 1.4 | 15.9 |
| 30% metaflumizone | % Efficacy | 100.0 | 98.9 | 99.4 | 98.4 | 97.5 | 73.1 |
| 120 mg/kg | | | | | | | |
| E | G.M. | 1.2 | 2.3 | 11.5 | 12.5 | 19.2 | 50.6 |
| 7.5 mg fipronil*/kg | % Efficacy | 97.7 | 96.4 | 78.8 | 81.1 | 66.9 | 14.3 |
| F | G.M. | 1.2 | 0.0 | 3.4 | 3.5 | 20.3 | 40.4 |
| 22.5 mg fipronil*/kg | % Efficacy | 97.8 | 100.0 | 93.8 | 94.7 | 64.9 | 31.5 |
| G | G.M. | 0.0 | 1.0 | 10.0 | 10.0 | 16.7 | 33.3 |
| 10 mg imidacloprid**/kg | % Efficacy | 100.0 | 98.5 | 81.4 | 84.8 | 71.1 | 43.5 |
| H | G.M. | 0.0 | 0.3 | 0.0 | 0.7 | 2.3 | 5.1 |
| 30 mg imidacloprid/kg | % Efficacy | 100.0 | 99.5 | 100.0 | 99.0 | 96.0 | 91.4 |

*The fipronil formulation used in the present Example was Frontline ®, as sold by Merial Limited (Duluth, GA)
**The imidacloprid formulation used in the present Example was Advantage ® as sold by Bayer Corporation (Shawnee Mission, KS)

### EXAMPLE 6

### Evaluation of the Duration of Control in Dogs Treated with Ectoparasiticides at Up to 3× the Conventional Dose Against the Cat Flea, Ctenocephalides felis

In this evaluation, 32 mixed breed male or female dogs, aged 1 to 10 years, were used. Dogs were not treated with an ectoparasiticide for 60 days prior to the study. No drugs, baths, shampoos or pesticides were given to the dogs during the preconditioning phase or during the course of the study other than what is described in the protocol. At least 36 dogs were preconditioned for 14 days. All dogs were bathed with noninsecticidal shampoo (Day −12 to −8). On Day −7, each dog was infested with 100 unfed cat fleas, Ctenocephalides felis. On Day −5, each dog was thoroughly examined and then combed to remove and count fleas. Dogs were selected for inclusion in the study on the basis of flea retention and/or behavior. The 32 dogs with the highest flea counts were selected for the study, except for exclusion of dogs deemed not easily handled. The dogs were blocked by flea number retained, and randomly allocated within blocks into 8 groups of 4 dogs. The selected dogs were weighed on Day −1 and received one of the flea treatments presented in Table 7.

TABLE 7
FLEA TREATMENTS APPLIED TO DOGS

| | | | Appli- | |
|---|---|---|---|---|
| | | | cation | Dose |
| | Number | | Rate | (mg/ |
| Group | of Dogs | Treatment | (ml/kg) | kg) |
| A | 4 | Control -- No treatment | 0.0 | 0 |
| B | 4 | 15% Metaflumizone | 0.13 | 20 |
| C | 4 | 30% Metaflumizone | 0.40 | 120 |
| D | 4 | 30% Metaflumizone | 0.20 | 60 |
| E | 4 | 10% imidacloprid** | 0.10 | 10 |
| F | 4 | 10% imidacloprid** | 0.30 | 30 |
| G | 4 | 10% fipronil* | 0.07 | 7 |
| H | 4 | 10% fipronil* | 0.21 | 21 |

*The fipronil formulation used in the present Example was Frontline ®, as sold by Merial Limited (Duluth, GA)
**The imidacloprid formulation used in the present Example was Advantage ® as sold by Bayer Corporation (Pittsburgh, PA)

Applications for Groups B-H were administered using disposable syringes. Each dog received a spot of the appropriate formulation to the skin (by syringe without a needle) at the dorsal midline between the shoulder blades. Group A was left untreated. The dogs were observed for any immediate reactions to the treatments. Observations for post-treatment adverse reactions, skin irritation and behavior of test formulations were made at four hours following administration of treatments. Thereafter, dogs were observed once daily on Days 1-14 for formulation acceptability, adverse reactions, or signs of skin irritation following treatment. Daily observations of the physical condition of the animals were made throughout the trial.

Dogs were infested with 100 adult cat fleas, Ctenocephalides felis, on Day 14 and then examined for live fleas on Day 16. The dogs were re-infested/examined on Days 28/30, 56/58, 70/72, 84/86 and then infested and examined again at 2 week intervals until efficacy waned. At each infestation, 100 unfed adult fleas were applied along the lateral midline of each dog. The negative control group (Group A) was infested before the test compound groups. Fleas were recovered by examining and combing the groups in the same order. Combs were washed and rinsed with alcohol between dogs. Plastic gloves and aprons were changed between treatment groups.

Flea counts were transformed by log (count +1), and geometric means were used to calculate percent efficacy for the treatments using (Yc−Yt)/Yc*100, where Yc and Yt equal mean counts for control and treated groups, respectively. The results are shown in Table 8 hereinbelow.

As shown in Table 8, the formulation providing the 3× (60 mg/kg) dosage (Group D) of metaflumizone was substantially more effective at Days 86, 100, 114 and 128 compared to the 1× (20 mg/kg; Group B) dosage (not tested on Days 114 and 128 due to low efficacy on prior days); the 3× dosage of metaflumizone was 96.5% effective at day 128. Similarly, the 3× (30 mg/kg, Group F) dosage of imidacloprid was substantially more effective at Days 72, 86, 100, 114 and 128 compared to the 1× (10 mg/kg, Group E) dosage; the 3× dosage of imidacloprid was 94.5% effective at Day 128.

TABLE 8
AVERAGE FLEA COMB COUNTS FOR DOGS ADMINISTERED HIGH DOSE
RATES OF METAFLUMIZONE, FIPRONIL, OR IMIDACLOPRID

| | | Flea Counts/Dog/Day (Geometric Means) | | | | | | | |
|---|---|---|---|---|---|---|---|---|---|
| Group¹ | | 16 | 30 | 58 | 72 | 86 | 100 | 114 | 128 |
| A Untreated Control | | 79.8 | 85.2 | 81.0 | 77.8 | 87.2 | 72.9 | 67.0 | 46.3 |
| B 15% MTF Spot-On | | 0.0 | 0.2 | 4.2 | 10.8 | 35.9 | 35.9 | NT² | NT |
| 20 mg/kg (1×) | % Efficacy | 100.0 | 99.8 | 94.8 | 86.1 | 58.8 | 50.8 | — | — |
| C 30% MTF Spot-On | ^{a} | 0.0 | 0.0 | 0.0 | 2.3 | 13.2 | 14.8 | 29.4 | NT |
| 120 mg/kg (6×) | % Efficacy | 100.0 | 100.0 | 100.0 | 97.1 | 84.9 | 79.7 | 56.2 | — |
| D 30% MTF Spot-On | | 0.0 | 0.0 | 2.5 | 0.6 | 1.3 | 1.4 | 1.2 | 1.6 |
| 60 mg/kg (3×) | % Efficacy | 100.0 | 100.0 | 96.9 | 99.3 | 98.5 | 98.1 | 98.3 | 96.5 |
| E 10 mg imidacloprid**/kg | | 0.0 | 0.0 | 1.5 | 15.4 | 9.7 | 15.8 | 31.7 | NT |
| (1×) | % Efficacy | 100.0 | 100.0 | 98.1 | 80.3 | 88.9 | 78.3 | 52.6 | — |
| F 30 mg imidacloprid**/kg | ^{a} | 0.0 | 0.0 | 0.0 | 2.4 | 3.1 | 2.3 | 2.3 | 2.5 |
| (3×) | % Efficacy | 100.0 | 100.0 | 100.0 | 97.0 | 96.4 | 96.8 | 96.6 | 94.5 |
| G 7 mg fipronil*/kg | | 0.2 | 0.2 | 0.0 | 2.4 | 2.3 | 4.3 | 10.2 | NT |
| (1×) | % Efficacy | 99.8 | 99.8 | 100.0 | 96.9 | 97.4 | 94.1 | 84.7 | — |
| H 21 mg fipronil*/kg | | 0.0 | 0.0 | 7.5 | 11.4 | 45.3 | 44.1 | NT | NT |
| (3×) | % Efficacy | 100.0 | 100.0 | 90.7 | 85.4 | 48.0 | 39.5 | — | — |

¹MTF = Metaflumizone
²NT = Not tested.
^{a}One dog died. N = 3 after Day 30.
*The fipronil formulation used in the present Example was Frontline ®, as sold by Merial Limited (Duluth, GA)
**The imidacloprid formulation used in the present Example was Advantage ® as sold by Bayer Corporation (Pittsburgh, PA)

### EXAMPLE 7

### Evaluation of the Duration of Efficacy of High Doses (1.5×, 3×, 4×, 5× and 6× the Conventional Dose) of Ectoparasiticides Against the Cat Flea, Ctenocephalides felis, in Cats

In this evaluation, 48 domestic shorthair cats of mixed sex, aged at least 6 months, were used to test the high doses of ectoparasiticides disclosed herein. Cats were free of ectoparasiticide treatment for 60 days prior to administration of the experimental treatments. No drugs, baths, shampoos, or pesticides were given to the cats during the preconditioning phase or during the course of the study other than what is described in the protocol. At least 48 cats were preconditioned for 14 days. On day −8, each cat was infested with 100 unfed cat fleas, Ctenocephalides felis. On Day −6, each cat was thoroughly examined and combed to remove and count fleas. 48 cats were ranked in descending order by flea count. From the ranking, the cats were divided into eight blocks of six cats each. The six cats within each of the eight blocks were randomly allocated to treatment groups (A-F). The cats were weighed on Day −1 and received one of the treatments presented in Table 9.

TABLE 9
FLEA TREATMENTS APPLIED TO CATS

| | | | Application | Metaflumizone |
|---|---|---|---|---|
| | No. | | Rate | Dose |
| Group | Cats | Treatment | (mL/kg BW) | (mg/kg BW) |
| A | 8 | Nontreated | 0 | 0 |
| B | 8 | 35% Metaflumizone | 0.171 | 60 |
| C | 8 | 35% Metaflumizone | 0.343 | 120 |
| D | 8 | 35% Metaflumizone | 0.457 | 160 |
| E | 8 | 35% Metaflumizone | 0.571 | 200 |
| F | 8 | 35% Metaflumizone | 0.686 | 240 |

Applications for groups B-F were administered using a disposable syringe. The dose was applied as a single spot on the dorsal neck at the base of the skull. Group A was left untreated. The cats were observed for any immediate reactions to the treatments, and were observed for post-treatment adverse reactions, skin irritation, and behavior of test formulations at the time of treatment, after approximately four hours. On Days 1 through 14 following administration of the treatments, cats were observed daily for reactions and formulation acceptability. Thereafter, cats were observed once daily for the remainder of the study. Cats were infested with 100 adult cat fleas on Day 14 then examined for fleas on Day 16. The cats were re-infested and examined again on Days 28 and 30, on Days 56 and 58, Days 70 and 72, Days 84 and 86, and Days 98 and 100. At each infestation, 100 unfed adult fleas were applied along the lateral midline of each cat. The negative control group (Group A) was infested before the test compound groups. Fleas were recovered by examining and combing the groups in the same order. Combs were washed and rinsed with alcohol between cats, and latex gloves were changed between treatment groups.

Flea counts were transformed by log (count +1), and geometric means were used to calculate percent efficacy for the treatments using (Yc−Yt)/Yc*100, where Yc and Yt equal mean counts for control and treated groups, respectively. The results are shown in Table 10 hereinbelow.

As shown in Table 10, cats treated with higher doses (5× or 6× the conventional dosage) of metaflumizone experienced sustained protection from fleas for longer periods of time compared to cats treated with lower doses.

TABLE 10
AVERAGE FLEA COMB COUNTS FOR CATS ADMINISTERED
HIGH-DOSE RATES OF METAFLUMIZONE

| Treatment Group | | Day 16 | Day 30 | Day 58 | Day 72 | Day 86 | Day 100 |
|---|---|---|---|---|---|---|---|
| A Untreated | G.M. | 43.8 | 65.7 | 55.0 | 56.3 | 55.7 | 33.3 |
| B 35% w/v | G.M. | 0.3 | 0.9 | 11.2 | 8.1 | 16.8 | 9.8 |
| Metaflumizone | | | | | | | |
| 60 mg ai/kg BW | % Efficacy | 99.2 | 98.6 | 79.7 | 85.6 | 69.8 | 70.7 |
| C 35% w/v | G.M. | 0.1 | 0.3 | 10.0 | 8.4 | 19.1 | 12.9 |
| Metaflumizone | | | | | | | |
| 120 mg ai/kg BW | % Efficacy | 99.8 | 99.5 | 81.9 | 85.1 | 65.8 | 61.1 |
| D 35% w/v | G.M. | 0.1 | 0.0 | 8.4 | 7.4 | 18.5 | 11.6 |
| Metaflumizone | | | | | | | |
| 160 mg ai/kg BW | % Efficacy | 99.8 | 100.0 | 84.7 | 86.9 | 66.7 | 65.3 |
| E 35% w/v | G.M. | 0.0 | 0.1 | 3.9 | 7.6 | 2.2 | 8.7 |
| Metaflumizone | | | | | | | |
| 200 mg ai/kg BW | % Efficacy | 100.0 | 99.9 | 92.8 | 86.5 | 96.1 | 73.8 |
| F 35% w/v | G.M. | 0.1 | 0.0 | 2.9 | 4.5 | 2.8 | 8.2 |
| Metaflumizone | | | | | | | |
| 240 mg ai/kg BW | % Efficacy | 99.8 | 100.0 | 94.7 | 92.0 | 94.9 | 75.4 |

### EXAMPLE 8

### Evaluation of the Duration of Control in Dogs Treated with Ectoparasiticides at 35% (W/V) Dose of Metaflumizone

In this evaluation, 40 adult male or female Beagle dogs, aged 2 to 9 years, were used. Dogs were not treated with an ectoparasiticide for 60 days prior to the study. No drugs, baths, shampoos or pesticides were given to the dogs during the preconditioning phase or during the course of the study other than what is described in the protocol. At least 42 dogs were preconditioned for 10 days. All dogs were bathed with noninsecticidal shampoo (Day −9). On Day −8, each dog was infested with 100 unfed cat fleas, Ctenocephalides felis. On Day −6, each dog was thoroughly examined and then combed to remove and count fleas. Dogs were selected for inclusion in the study on the basis of flea retention and/or behavior. The 40 dogs with the highest flea counts were selected for the study. The dogs were blocked by flea number retained, and randomly allocated within blocks into 5 groups of 8 dogs. The selected dogs were weighed on Day −1 and received one of the flea treatments presented in Table 11.

TABLE 11
FLEA TREATMENTS APPLIED TO DOGS

| | | | Appli- | |
|---|---|---|---|---|
| | | | cation | Dose |
| | Number | | Rate | (mg/ |
| Group | of Dogs | Treatment | (ml/kg) | kg) |
| A | 8 | Control -- No treatment | 0.0 | 0 |
| B | 8 | 35% Metaflumizone (Dog Formula) | 0.086 | 30 |
| C | 8 | 35% Metaflumizone (Dog Formula) | 0.171 | 60 |
| D | 8 | 35% Metaflumizone (Dog Formula) | 0.343 | 120 |
| E | 8 | 35% Metaflumizone (Cat Formula) | 0.171 | 60 |

Applications for Groups B-E were administered using disposable syringes. Each dog received a spot of the appropriate formulation to the skin (by syringe without a needle) at the dorsal midline between the shoulder blades. Group A was left untreated. The dogs were observed for any immediate reactions to the treatments. Observations for post-treatment adverse reactions, skin irritation and behavior of test formulations were made at approximately hourly intervals for four hours following treatment of the last dog. Thereafter, dogs were observed once daily until the final flea count. Daily observations of the physical condition of the animals were made throughout the trial.

Dogs were infested with 100 adult cat fleas, Ctenocephalides felis, on Day 14 and then examined for live fleas on Day 16. The dogs were re-infested/examined on Days 28/30, 56/58 and then infested and examined again at 2 week intervals until efficacy waned. At each infestation, 100 unfed adult fleas were applied along the lateral midline of each dog. The negative control group (Group A) was infested before the test compound groups. Fleas were recovered by examining and combing the groups in the same order. Combs were washed and rinsed with alcohol between dogs. Plastic gloves and aprons were changed between treatment groups.

Flea counts were transformed by log (count +1), and geometric means were used to calculate percent efficacy for the treatments using (Yc−Yt)/Yc*100, where Yc and Yt equal mean counts for control and treated groups, respectively. The results are shown in Table 12 hereinbelow.

TABLE 12
AVERAGE FLEA COMB COUNTS FOR DOGS ADMINISTERED
HIGH DOSE RATES (35%) OF METAFLUMIZONE

| | | Flea Counts/Dog (Geometric Means) | | | | | | | | | |
|---|---|---|---|---|---|---|---|---|---|---|---|
| | | | Day | Day | Day | Day | Day | Day | Day | Day | Day |
| Group | | Day −6 | 16 | 30 | 58 | 72 | 86 | 100 | 114 | 128 | 142 |
| A Control/ | | 77.0 | 69.9 | 74.2 | 70.5 | 75.5 | 75.9 | 76.6 | 70.7 | 70.0 | 71.9 |
| Untreated | | — | | | | | | | | | |
| B 30 mg/kg | | 76.5 | 0.1 | 0.0 | 0.4 | 0.8 | 2.9 | 5.9 | 5.0 | 10.4 | 13.4 |
| Dog Formula | % Eff | — | 99.9 | 100.0 | 99.4 | 99.0 | 96.2 | 92.3 | 92.9 | 85.2 | 81.4 |
| C 60 mg/kg | | 75.3 | 0.4 | 0.1 | 0.7 | 1.9 | 4.4 | 7.9 | 6.4 | 12.4 | 13.3 |
| Dog Formula | % Eff | — | 99.5 | 99.9 | 99.1 | 97.5 | 94.2 | 89.6 | 90.9 | 82.3 | 81.5 |
| D 120 mg/kg | | 77.3 | 0.1 | 0.0 | 0.0 | 0.0 | 0.1 | 0.5 | 1.2 | 4.4 | 8.7 |
| Dog Formula | % Eff | — | 99.9 | 100.0 | 100.0 | 100.0 | 99.9 | 99.4 | 98.3 | 93.7 | 87.9 |
| E 60 mg/kg | | 78.3 | 0.0 | 0.0 | 0.4 | 0.7 | 1.4 | 2.0 | 3.8 | 5.0 | 7.8 |
| Cat Formula | % Eff | — | 100.0 | 100.0 | 99.5 | 99.0 | 98.1 | 97.4 | 94.6 | 92.9 | 89.2 |

Treatment of dogs with 35% w/v metaflumizone spot-on formulations at 30 to 120 mg/kg BW provided at least 90% residual flea control for 114 days (30 and 60 mg/kg dose) to 128 days (120 mg/kg and 60 mg/kg cat formula).

### EXAMPLE 9

### Dosing Formulations

Tables 13 and 14 show amounts of active ingredients for metaflumizone-based formulations for dogs (Table 13) and cats (Table 14)

TABLE 13

| | Metaflumizone (a.i.) Dog Formulation | | | | |
|---|---|---|---|---|---|
| Body Wt (kg) | <5 | 5-10 | 10-20 | 20-40 | 40-60 |
| Volume of | 1 | 2 | 4 | 8 | 12 |
| composition (mL) | | | | | |
| Dose of a.i. (g) | 0.3 | 0.6 | 1.2 | 2.4 | 3.6 |
| Conc. of a.i. (% w/v) | 30.0 | 30.0 | 30.0 | 30.0 | 30.0 |

TABLE 14

| | Metaflumizone (a.i.) Cat Formulation | |
|---|---|---|
| Body Wt (kg) | <4 | 4-8 |
| Volume of composition (mL) | 1.6 | 3.2 |
| Dose of a.i. (g) | 0.48 | 0.96 |
| Conc. of a.i. (% w/v) | 30.0 | 30.0 |

### EXAMPLE 10

### Dosing and Application of Compositions Containing Pyriprole

The coat on the back of the animal is parted between the shoulder blades until the skin is visible. The tip of a pipette containing pyriprole is applied on the skin and squeezed gently several times at one or two spots, thereby emptying the contents onto the skin. For large dogs additional spots may be applied down the back so as to prevent run-off.

In Table 15B, a minimum dose of 30 mg/kg bodyweight pyriprole is used. While a constant concentration of pyriprole (25% w/v) is shown in Table 15A, additional concentrations, such as 20%, 21%, 22%, 23%, 24%, 26%, 27%, 28%, 29% and 30% are also contemplated for use herein.

TABLE 15A

| | |
|---|---|
| | Volume of 25% |
| Weight of dog | (w/v) |
| (kg) | solution (ml) |

| | | |
|---|---|---|
| 2-4.5 | kg | 0.5-1 |
| >4.5-11 | kg | 1.5-2.5 |
| >11-22 | kg | 3-4.5 |
| >22-50 | kg | 5-9 |
| More than 50 | kg | 6-12 |

TABLE 15B

| | |
|---|---|
| Weight of dog | Pyriprole |
| (kg) | (mg/kg bw) |

| | | |
|---|---|---|
| 2-4.5 | kg | 30-60 |
| >4.5-11 | kg | 30-70 |
| >11-22 | kg | 30-80 |
| >22-50 | kg | 30-90 |
| More than 50 | kg | 30-120 |

It will be apparent to those skilled in the art that various modifications and variation can be made to the compositions and methods described herein and as provided in the examples above without departing from the spirit or scope of the compositions and methods.

All cited patents and publications referred to in this application are herein incorporated by reference in their entirety.

What is claimed is:

1. A method for preventing or treating an ectoparasite infestation in a warm-blooded animal for a period greater than 6 weeks, comprising:
topically administering to the warm-blooded animal a composition comprising an ectoparasiticide in a dose that is about 1.5 to 6 times the amount of a conventional dose for said ectoparasiticide.

2. The method of claim 1, wherein said period is from 8 to 20 weeks.

3. The method of claim 1, wherein said ectoparasiticide is metaflumizone, fipronil, imidacloprid, pyriproledinotefuran or a mixture thereof.

4. The method of claim 1, wherein said animal is a dog or a cat.

5. The method of claim 1, wherein said ectoparasiticide is metaflumizone.

6. The method of claim 5, wherein said animal is a dog and said dose is about 35-120 mg metaflumizone per kg of body weight.

7. The method of claim 6, wherein said dose is about 35-100 mg metaflumizone per kg of body weight.

8. The method of claim 6, wherein said dose is about 40-80 mg metaflumizone per kg of body weight.

9. The method of claim 5, wherein the composition further comprises amitraz.

10. The method of claim 5, wherein said animal is a cat and said dose is about 60-240 mg metaflumizone per kg of body weight.

11. The method of claim 10, wherein said dose is about 70-160 mg metaflumizone per kg of body weight.

12. The method of claim 10, wherein said dose is about 80-160 mg metaflumizone per kg of body weight.

13. The method of claim 1, wherein said ectoparasiticide is fipronil and said dose is about 10-50 mg fipronil per kg of body weight.

14. The method of claim 13, wherein said dose is about 15-35 mg fipronil per kg of body weight.

15. The method of claim 13, wherein the composition further comprises methoprene.

16. The method of claim 1, wherein said ectoparasiticide is imidacloprid and said dose is about 15-60 mg imidacloprid per kg of body weight.

17. The method of claim 16, wherein said dose is about 20-50 mg imidacloprid per kg of body weight.

18. The method of claim 16, wherein the composition further comprises permethrin.

19. The method of claim 1, wherein the ectoparasiticide is in a unit dose 1.5 to 5 times more concentrated than the conventional dose for said ectoparasiticide.

20. The method of claim 5, wherein the composition comprises 26%-40% w/v metaflumizone.

21. The method of claim 5, wherein the composition comprises about 30% w/v metaflumizone.

22. The method of claim 1, wherein the volume of the composition per animal body weight is from 0.1 mL/kg to 1 mL/kg.

23. The method of claim 1, wherein the composition further comprises at least one of a bridging agent, a surfactant or a carrier solvent.

24. The method of claim 1, wherein the composition comprises about 30% w/v metaflumizone, dimethylsulfoxide and γ-hexalactone.

25. A method for preventing or treating an ectoparasite infestation in a warm-blooded animal for a period greater than 6 weeks, comprising:
topically administering to the warm-blooded animal a composition comprising on a weight by volume basis:
(a) about 5% to about 40% of ectoparasiticide;
(b) about 5% to about 25% of a bridging agent;
(c) about 0% to about 15% of a surfactant; and
(d) about 5% to about 80% of a carrier solvent or a mixture of solvents;
wherein the ectoparasiticide is in a dose that is about 1.5 to 5 times the amount of a conventional dose for said ectoparasiticide.

26. The method of claim 25, wherein the ectoparasiticide is metaflumizone.

27. The method of claim 26, wherein the composition further comprises amitraz.

28. The method of claim 26 wherein the carrier solvent comprises γ-hexalactone.

29. The method of claim 28, wherein the carrier solvent further comprises N,N-diethyl-m-toluamide.

30. The method of claim 28, wherein the carrier solvent further comprises 1-methoxy-2-propyl acetate.

31. The method of claim 28, wherein the carrier solvent further comprises eucalyptol.

32. The method of claim 26, wherein the bridging agent is dimethylsulfoxide (DMSO).

33. A composition comprising on a weight to volume basis:
(a) 26% to about 40% of metaflumizone;
(b) about 4% to about 25% of a bridging agent;
(c) about 0% to about 15% of a surfactant; and
(d) about 5% to about 70% of a carrier solvent or a mixture of solvents.

34. The composition of claim 33, wherein metaflumizone is present at about 30% on a weight to volume basis.

35. The composition of claim 33, wherein said bridging agent is selected from the group consisting of an alkyl methylsulfoxide, dimethylsulfoxide (DMSO), decylmethyl sulfoxide, tetradecylmethyl, sulfoxide, a pyrrolidone, 2-pyrrolidone, N-methyl-2-pyrrolidone, N-(2-hydroxyethyl)pyrrolidone, a laurocapram, a solvent, acetone, dimethyl acetamide, dimethylformamide, and tetrahydrofurfuryl alcohol.

36. The composition of claim 33, wherein the bridging agent is selected from the group consisting of an L-amino acid, dimethylsulfoxide (DMSO) and a fatty acid.

37. The composition of claim 33, wherein the surfactant is selected from the group consisting of an alcohol alkoxylate surfactant, a nonylphenol ethoxylate, an anionic or cationic surfactant, and a non-ionic surfactant.

38. The composition of claim 33, wherein the carrier solvent is selected from the group consisting of a diluent, an adjuvant, an excipient, a preservative, and a vehicle with which a compound or composition is administered.

39. The composition of claim 33, wherein said carrier solvent is selected from the group consisting of petroleum oil, animal oil, vegetable oil, peanut oil, soybean oil, mineral oil, and sesame oil.

40. The composition of claim 33, wherein said carrier solvent comprises γ-hexalactone.

41. The composition of claim 40, further comprising a second carrier solvent selected from the group consisting of N,N-diethyl-m-toluamide, eucalyptol, dimethyl isosorbide, diisopropyl adipate, and 1-methoxy-2-propyl acetate.

42. The composition of claim 33, comprising, on a weight to volume basis, 27% to 35% metaflumizone; about 10% of the bridging agent dimethyl sulfoxide; and between about 45% and about 60% of the carrier solvent γ-hexalactone.

43. The composition of claim 33, further comprising a preservative selected from the group consisting of methylparaben, propylparaben, thimerosol, and EDTA.

44. The composition of claim 33, further comprising a polymeric agent.

45. The composition of claim 33, wherein the ectoparasiticide is present in a concentration of 10-50 mg/mL.

46. The composition of claim 33, wherein the total volume of the composition is less than about 13 mL.

47. The composition of claim 33, further comprising a polymeric agent selected from the group consisting of colloidal silicone dioxide, ethyl cellulose, methyl cellulose, a methacrylic ester copolymer, a carboxylated vinyl acetate terpolymer, a polyvinylpropylene (PVP)/Vinyl acetate copolymer, polyvinylmethylether, poly(vinylmethylether/maleic anhydride, an ethyl or butyl ester of polyvinylmethylether/maleic anhydride copolymer, and an ethyl or butyl ester of a PVM/MA copolymer.

48. The composition of claim 33, further comprising amitraz.

49. A kit for preventing or treating an ectoparasite infestation in a warm blooded animal comprising a topical unit dose formulation of an ectoparasiticide comprising on a weight by volume basis:
(a) about 5% to about 40% of an ectoparasiticide;
(b) about 5% to about 25% of a bridging agent;
(c) about 0% to about 15% of a surfactant; and
(d) about 5% to about 80% of a carrier solvent or a mixture of solvents;
wherein the unit dose comprises about 1.5 to 5 times the amount of the ectoparasiticide as compared to a conventional dose of said ectoparasiticide.

50. The kit of claim 49, wherein the ectoparasiticide is metaflumizone, fipronil, imidacloprid, pyriprole, dinotefuran or a mixture thereof.

51. The kit of claim 49, wherein said ectoparasiticide is metaflumizone, said animal is a dog and said unit dose comprises between about 40 mg and about 120 mg metaflumizone per kg of body weight of the dog to which said composition is to be administered.

52. The kit of claim 51, wherein the formulation further comprises amitraz.

53. The kit of claim 49, wherein said ectoparasiticide is metaflumizone, said animal is a cat and said single dose comprises between about 60 mg and about 240 mg metaflumizone per kg of body weight of the cat to which said composition is to be administered.

54. The kit of claim 49, wherein said ectoparasiticide is imidacloprid, said animal is a cat or dog and said single dose comprises between about 15 mg and about 100 mg imidacloprid per kg of body weight of the cat or dog to which said composition is to be administered.

55. The kit of claim 54, wherein said animal is a dog and said formulation further comprises permethrin.

56. The kit of claim 49, wherein said ectoparasiticide is fipronil, said animal is a cat or dog and said single dose comprises between about 10 mg and about 100 mg fipronil per kg of body weight of the cat or dog to which said composition is to be administered.

57. The kit of claim 55, wherein the animal is a dog and the formulation further comprises methoprene.

58. The kit of claim 49, wherein said unit dose is effective in preventing or treating the ectoparasite infestation in said warm-blooded animal for a period greater than about 6 weeks.
